(19) United States
(12) Patent Application Publication
(10) Pub. No.: US 2020/0078393 A1
(43) Pub. Date: Mar. 12, 2020

(54) COMPOSITION COMPRISING HYDROGEN GAS FOR SUPPRESSION OR PREVENTION OF CANCER METASTASIS

(71) Applicant: MiZ Company Limited, Kanagawa (JP)

(72) Inventors: Fumitake Satoh, Kanagawa (JP); Shinichi Hirano, Kanagawa (JP); Ryosuke Kurokawa, Kanagawa (JP); Yusuke Ichikawa, Kanagawa (JP); Takehisa Nakayama, Saitama (JP)

(21) Appl. No.: 16/568,004

(22) Filed: Sep. 11, 2019

(30) Foreign Application Priority Data
Sep. 12, 2018 (JP) 2018-170579
Mar. 25, 2019 (JP) 2019-056298

(51) Int. Cl. A61K 33/00; A61K 9/00; A61P 35/04

(52) U.S. Cl. CPC A61K 33/00; A61P 35/04; A61K 9/007

(57) ABSTRACT

This application provides a composition for suppressing or preventing metastasis of a cancer, or for suppressing growth of an end stage cancer with metastasis and/or a metastatic cancer(s) therefrom, in a subject having a cancer, the composition comprising gaseous hydrogen as an active ingredient, as well as a method for suppressing or preventing metastasis or suppressing growth of an end stage cancer with metastasis and/or a metastatic cancer(s) therefrom in a subject, comprising administering this composition to the subject having a cancer.

### FIELD OF THE INVENTION

The present invention relates to a composition comprising gaseous hydrogen for suppressing or preventing metastasis of a cancer.

The present invention also relates to a composition for suppressing growth of an end stage cancer with metastasis and a metastatic cancer(s) therefrom, the composition comprising gaseous hydrogen as an active ingredient.

### BACKGROUND ART

The number of deaths due to cancers in Japan, according to a report of National Cancer Center Japan (Tokyo, Japan), exceeded 370,000 in 2017, which is recognized as a social problem. Lifestyle such as smoking and drinking alcohol and infection with human papillomavirus, Helicobacter pylori, and the like, are noted as causes of cancers. At least 81 carcinogens are involved with cancers such as a lung cancer caused by smoking, and it is documented that these carcinogens, in addition to working as initiators, also work as carcinogenic promoters by triggering chronic inflammation (Non-Patent Document 1).

In cancer patients, metastasis is likely to occur as a cancer progresses and thus a survival rate of patients with an end stage cancer with metastasis notably decreases. Additionally, the whole body is affected by a primary cancer and a metastatic cancer(s) therefrom, causing a patient to suffer from intense pains together with notably decreased QOL (Quality Of Life) due to adverse reactions to an anticancer agent administered for a therapy.

Once cancer cells are developed and tumors are formed, both normal cells present close to tumors and cancer cells initiate an inflammation response associated with the progress to malignancy. In other words, cancer cells secret chemoattractants called chemokines to recruit white blood cells such as Tumor-Associated Macrophage (TAM). TAM produces a tumor necrosis factor α (TNF-α), which is a kind of the cytokines. This cytokine induces molecules acting to maintain the inflammation response and helps the inflammation response organization. TAM also produces ROS and RNS in addition to the cytokine and DNA damages caused by ROS and RNS allow tumor growth to proceed (Non-Patent Document 2).

Surgical therapy, drug therapy using an anticancer agent such as a molecularly targeted drug, and radiotherapy are mainly known as three major therapies for cancer treatment methods.

Surgical therapy is a method of surgically excising cancer lesions. This treatment method can remove a mass of cancer at once and thus offers a high potential for complete cure unless there is micrometastasis undetectable by examinations, hence beneficial. However, surgical therapy is associated with serious physical burden and mental burden on a patient. Further, when the body of a patient is incised to excise a lesion in a cancer surgery, ROS and RNS produced from white blood cells for infection protection may trigger inflammation, hence problematic.

Drug therapy is a method of administering an anticancer agent or the like, by internal use or injection to suppress growth of cancer cells. However, most of the anticancer agents are agents having strong toxicity and damage normal cells present in lesions other than cancer cells and also suppress the growth of normal cells thereby to trigger serious adverse reactions to the whole body, hence problematic.

For example, gefitinib (Iressa®) is known as a targeted drug effective on lung cancers. However, Iressa is known to have an adverse reaction that triggers interstitial pneumonia by damaging the alveolar epithelium. The adverse reaction as such became a social problem (Non-Patent Document 3).

Radiotherapy is to suppress growth of cancer cells by damaging the cancer cells by irradiating lesions with radiation. However, when irradiating lesions with radiation, normal cells present nearby the lesions are also irradiated with radiation causing adverse reactions such as cell damages including inflammation, hence problematic.

Inflammation can be a cause of cancers as described above and may be caused by any of conventional three major cancer therapies. For this reason, it is important to prevent the occurrence of inflammation for preventing cancers and thus a therapy capable of suppressing inflammation is in demand for treating cancers.

Yanagihara et al. documented that electrolyzed hydrogen water suppresses oxidative damages in rats (Non-Patent Document 4). Thereafter, Oosawa et al. documented that oxidative stress in the brain triggered by a large amount of reactive oxygen species caused by ischemia-reperfusion injury is suppressed by the suction of gaseous hydrogen in a concentration of 2% to 4%, whereby hydrogen selectively reduces ROS and RNS representing cytotoxicity and thus works as an antioxidant effective for a therapy (Non-Patent Document 5). Ever since these documents, gaseous hydrogen has attracted attentions as an antioxidant substance which easily spreads in vivo when sucked and selectively reduces hydroxyl radical (.OH) and peroxynitrite (ONOO⁻) particularly highly reactive among the reactive oxygen species.

There is a document on the therapeutic effect of gaseous hydrogen against cancers which exhibits the suppression effect of electrolyzed water containing platinum nano colloid comprising hydrogen produced by electrolysis on cancer cells (Non-Patent Document 6). A further document indicates cell killing effect of palladium-nickel based hydrogen storage alloys against cancer cells (Non-Patent Document 7). Furthermore, a document presents that suction of gaseous hydrogen relieves renal toxicity, which is an adverse reaction to cisplatin known as an anticancer agent, without deteriorating the anticancer activity of cisplatin (Non-Patent Document 8). However, all these documents are from tests which used cultured cells and not from tests in which gaseous hydrogen was used alone or observed the effects of in vivo experiments. For this reason, the actual effectiveness on animals is not known and possible effects of other substances coexisting with gaseous hydrogen cannot be denied. Typically, cultured cells and tissues of an animal individual exist in different environments and results obtained from cultured cells are not always applicable directly to tissues of an animal individual. Thus, effects of gaseous hydrogen on cancer cells need to be tested at the individual level.

Effects of hydrogen on cancers by gaseous hydrogen inhalation are documented that 97.5% of the hydrogen inhalation suppressed adenocarcinomas transplanted to the skin of mice at an atmospheric pressure of 8 (Non-Patent Document 9). Additionally, Akagi et al. documented that, in intrahepatic cholangiocarcinomas, breast cancers, ovarian cancers, lung cancers, and pancreatic cancers, the inhalation of gaseous hydrogen demonstrates a cancer growth suppression effect by the mechanism of increasing cytotoxic T cells which exterminate cancer cells (Non-Patent Document 10). Further, Patent Document 1 discloses use of water comprising gaseous hydrogen dissolved therein for preventing, suppressing metastasis of, or preventing recurrence of upper digestive cancers, respiratory cancers, or body surface cancers, and in the disclosure of which such water has a blocking effect on cancer cell infiltration based on test results of culturing a fibrosarcoma cell line in gaseous hydrogen dissolved water-containing medium, however such an infiltration effect is not clear from the disclosure of this document. Consequently, there is no example documented to have verified that gaseous hydrogen singly has an effect for suppressing metastasis of a cancer based on in vivo experiments using highly metastatic animal models.

### PRIOR ART DOCUMENT

### Patent Document

[Patent Document 1] Japanese Patent Laid-Open No. 2007-254435 (JP 2007-254435 A)

### Non-Patent Document

[Non-Patent Document 1] Takahashi et al. Cancer cell, 2010; 17:89-97

[Non-Patent Document 2] Lauren Pecorino, co-translated by Hiroshi Hiai and Ryo Kominami, Molecular Biology of Cancer, 2^{nd} edition, by Pecorino, 2014, Medical Science International

[Non-Patent Document 3] Hiroshi Nojima, An Illustrated Guide to Cancer and Gene, 2009, Kodansha (Tokyo, Japan)

[Non-Patent Document 4] Tomoyuki Yanagihara et al., Biosci. Biotechnol. Biochem., 2005; 69(10):1985-1987

[Non-Patent Document 5] Ikuroh Osawa et al., Nature Medicine, 2007; 13:688-694

[Non-Patent Document 6] Yasukazu Saitoh et al., Oncol Res., 2008; 17:247-255

[Non-Patent Document 7] Yasukazu Saitoh et al., Exp Oncol, 2009; 31:156-162

[Non-Patent Document 8] Nakashima-Kanamori N et al., Cancer Chemother Pharmacol., 2009; 64(4):753-761

[Non-Patent Document 9] Malcome Dole et al., Science, 1975; 190:152-154

[Non-Patent Document 10] General Incorporated Association Japan Advanced Medical Treatment Clinical Application Society, Hydrogen Medical Treatment Group “Immunological effect of a hydrogen gas”, Junji Akagi (Director, TAMANA REGIONAL HEALTH MEDICAL CENTER), December, 2016

### SUMMARY OF THE INVENTION

### Problem to be Solved by the Invention

An object of the present invention is to suppress or prevent metastasis of a cancer, or to suppress growth of an end stage cancer with metastasis and/or a metastatic cancer(s) therefrom, in a subject by administering the composition comprising gaseous hydrogen as an active ingredient to the subject (or patient) having a cancer.

### Means for Solution of Problem

The present inventors conducted extensive studies to solve the above problems and found that when the composition comprising gaseous hydrogen according to the present invention is administered to a subject having a cancer, metastasis of the cancer can be suppressed or prevented as well as growth of an end stage cancer with metastasis and/or a metastatic cancer(s) therefrom in a subject is suppressed, whereby the present invention was accomplished.

The present invention includes the following features.

(1) A composition for suppressing or preventing metastasis of a cancer, or for suppressing growth of an end stage cancer with metastasis and/or a metastatic cancer(s) therefrom, in a subject having a cancer, wherein the composition comprises gaseous hydrogen as an active ingredient.
(2) The composition according to (1), wherein the gaseous hydrogen is diluted with air and/or oxygen.
(3) The composition according to (1) or (2), wherein the composition is to be sucked or inhaled by a human or an animal.
(4) The composition according to any of (1) to (3), wherein the cancer is selected from the group consisting of lung cancers, hepatic cancers, pancreatic cancers, stomach cancers, bile duct cancers, colorectal cancers, rectal cancers, bone cancers, prostate cancers, breast cancers, bladder cancers, urothelial cancers, esophageal cancers, brain tumors, ovarian cancers, cervical cancers, lymphomas, tongue cancers, head and neck cancers, osteosarcomas, skin cancers, myelomas, endometrial cancers, pharyngeal cancers, maxillary cancers, oral cancers, lip cancers, thyroid cancers, malignant melanomas, soft tissue tumors, angiosarcomas, undifferentiated soft tissue sarcomas, pediatric solid tumors, and leukemias.
(5) The composition according to any of (1) to (4), wherein the subject is a human.
(6) The composition according to any of (1) to (5), which is used in combination with any of other cancer therapies.
(7) A method for suppressing or preventing metastasis of a cancer in a subject, comprising administering the composition according to any of (1) to (6) to the subject having a cancer.
(8) A method for suppressing growth of an end stage cancer with metastasis and/or a metastatic cancer(s) therefrom in a subject, comprising administering the composition according to any of (1) to (6) to the subject having an end stage cancer with metastasis and/or a metastatic cancer(s) therefrom.

### Effect of the Invention

According to the present invention, when gaseous hydrogen (also called “hydrogen gas”, “molecular hydrogen”, “hydrogen molecules” or “hydrogen”) is administered to (for example, sucked or inhaled by) a subject (or patient) having a cancer, metastasis of the cancer can be suppressed or prevented or growth of an end stage cancer with metastasis and/or a metastatic cancer(s) therefrom can be suppressed.

### BRIEF DESCRIPTION OF THE DRAWINGS

FIG. 1 is photomicrographs (magnification, ×40) of lung tissue specimens of mice sacrificed 22 days after lung cancer cell transplantation to the lung of control group mice which did not inhale hydrogen gas-containing air. The above lung cancer cell is a highly metastatic mouse Lewis Lung Carcinoma cell line. In the figure, panels A and B show high engraftment levels of the lung cancer cell line (in the figure, the parts enclosed with an oval) into the lung tissues of different control mice tested;

FIG. 2 is photomicrographs (magnification, ×40) of lung tissue specimens of hydrogen-inhaled group mice which were kept in the air for 7 days after cancer cell transplantation into the mouse lung, then inhaled hydrogen gas-containing air for 1 hour a day for 4 days, and then sacrificed. The lung cancer cell is a highly metastatic mouse Lewis Lung Carcinoma cell line. In the figure, panels A and B show engraftment levels of the lung cancer cell line (in the figure, the parts enclosed with an oval) into the lung tissues of different mice tested. These results show low engraftment levels of the lung cancer cell line as the color of parts enclosed with an oval is lighter than the control group of FIG. 1;

FIG. 3 is photomicrographs (magnification, ×40) of lymph node tissue specimens of mice sacrificed 22 days after lung cancer cell transplantation to the lung of control group mice which did not inhale hydrogen gas-containing air. The above lung cancer cell is a highly metastatic mouse Lewis Lung Carcinoma cell line. The figure shows that the lung cancer cell line (the part in a dark color) has metastasized into the lymph node;

FIG. 4 is photomicrographs (magnification, ×200) of lung tissue specimens of mice sacrificed 22 days after lung cancer cell transplantation to the lung of control group mice which did not inhale hydrogen gas-containing air (A) and hydrogen-inhaled group mice which were kept in the air for 7 days after cancer cell transplantation into the mouse lung, then inhaled a hydrogen gas for 1 hour a day for 14 days, and then sacrificed (B). The above lung cancer cell is a highly metastatic mouse Lewis Lung Carcinoma cell line. In the figure, panels A and B show the difference in cell densities of the lung cancer cell line in the mouse lung tissues by presence or absence of the hydrogen gas inhalation; and

FIG. 5 is photomicrographs (magnification, ×400) of lung tissue specimens of different mice sacrificed 22 days after lung cancer cell transplantation to the lung of control group mice which did not inhale hydrogen gas-containing air (A) and hydrogen-inhaled group mice which were kept in the air for 7 days after cancer cell transplantation into the mouse lung, then inhaled a hydrogen gas for 1 hour a day for 14 days, and then sacrificed (B) (both are the same specimens as FIG. 4). The above cancer cell is a highly metastatic mouse Lewis Lung Carcinoma cell line. In the figure, panels A and B show the difference in cell densities of the lung cancer cell line in the mouse lung tissues by presence or absence of the hydrogen gas inhalation.

### MODES FOR CARRYING OUT THE INVENTION

The present invention is described in further detail.

The composition of the present invention comprises gaseous hydrogen as an active ingredient and, when administered to a subject having a cancer, can suppress or prevent metastasis of the cancer, or suppress growth of an end stage cancer with metastasis and/or a metastatic cancer(s) therefrom.

Hydrogen, due to the nature distinctive mainly in gaseous molecules, is conceived to spread and be distributed to various tissues including the brain and be also partially distributed to the whole body through blood stream thereby to relieve the inflammation of tissues invaded by cancer cells.

As used herein, the term “gaseous hydrogen”, an active ingredient, refers to, in the molecular formula, H₂, D₂ (deuterium), HD (deuterated hydrogen), or mixed gases thereof. D₂, although expensive, is known to have a stronger superoxide scavenging action than H₂. Hydrogens usable in the present invention are H₂, D₂ (deuterium), HD (deuterated hydrogen), or mixed gases thereof, and preferably H₂, or D₂ and/or HD may also be used in place of H₂ or by mixing with H₂.

The composition of the present invention can be, as described below, in the form of a gas or liquid comprising gaseous hydrogen.

Examples of the gas containing gaseous hydrogen include air containing gaseous hydrogen or gaseous oxygen containing gaseous hydrogen. The concentration of gaseous hydrogen is more than zero (0) and 18.5% by volume or less, for example, any concentrations specified in a range from about 0.1 to about 15.8% by volume, and preferably about 1 to about 10% by volume, about 1 to about 9% by volume, about 1 to about 8% by volume, about 1 to about 7% by volume, about 1 to about 6% by volume, about 1 to about 5% by volume, about 2 to about 10% by volume, about 2 to about 9% by volume, about 2 to about 8% by volume, about 2 to about 7% by volume, about 3 to about 10% by volume, about 3 to about 8% by volume, about 3 to about 7% by volume, about 3 to about 6% by volume, about 3 to about 4% by volume, about 4 to about 10% by volume, about 4 to about 8% by volume, about 4 to about 6% by volume, about 5 to about 10% by volume, about 5 to about 8% by volume, about 6 to about 10% by volume, and about 6 to about 8% by volume. The gaseous hydrogen is a flammable and explosive gas and thus must be contained in the composition so as to be a safe level to a subject such as human and administered to the subject.

When gases other than the gaseous hydrogen are the air, the concentration of air can be any concentration in the range from, for example, about 84.2 to about 99.9% by volume. Additionally, when gases other than the gaseous hydrogen include oxygen, the concentration of oxygen can be any concentration in the range from, for example, about 21 to about 99.9% by volume. Furthermore, the composition of the present invention can also contain gaseous nitrogen in addition to the gaseous oxygen.

The gaseous hydrogen, when administered to a subject, can be produced using a hydrogen gas supply device designed to be safe. The hydrogen gas supply device is not limited and, for example, enables gaseous hydrogen generated by the reaction of a hydrogen generating agent (for example, metal aluminum) and water to be mixed with a gas for dilution (for example, air and oxygen) in a predetermined ratio (Japanese Patent No. 5228142). Alternatively, gaseous hydrogen generated by using electrolysis of water can also be diluted and mixed (Japanese Patent No. 5502973 and Japanese Patent No. 5900688). According to these methods, a composition comprising gaseous hydrogen in a hydrogen concentration of, for example, 0.1 to 18.5% by volume can be prepared. Alternatively, a gas obtained by mixing a predetermined amount of a purified hydrogen gas and a purified air or a purified oxygen gas may also be used.

Alternatively, using a non-destructive hydrogen adding apparatus, specifically, an apparatus or equipment by which hydrogen molecules are added from the outside of a package of a commercial solution for living bodies such as infusion solutions (for example, enclosed in a hydrogen permeable plastic bag such as a polyethylene bag) (for example, sold by MiZ Company Limited (www.e-miz.co.jp/technology.html)), a bag containing a solution for living bodies is immersed in saturated hydrogen water to allow hydrogen to permeate into the bag thereby to dissolve hydrogen aseptically in the solution for living bodies until an equilibrium concentration is reached. This apparatus is constituted by, for example, an electrolyzer and a water tank and the water in the water tank can circulate between the electrolyzer and the water tank thereby to produce hydrogen by electrolysis. Alternatively, a simplified disposable equipment can be used for the same purpose (Japanese Patent Laid-Open No. 2016-112562 and the like). This equipment is internally provided with a solution for living body-containing plastic bag (hydrogen permeable bag, for example, a polyethylene bag) and a hydrogen generating agent (for example, metallic calcium, metallic magnesium/cation exchange resin) in an aluminum bag, and hydrogen generating agent is wrapped with, for example, non-woven fabric (for example, water vapor permeable non-woven cloth). The hydrogen generating agent wrapped with non-woven fabric is wetted with a small amount of water such as water vapor thereby to allow generated hydrogen to permeate through the plastic bag to be dissolved in the solution for living bodies non-destructively and germfree.

A hydrogen-dissolved liquid, or a liquid comprising gaseous hydrogen, is an aqueous liquid in which a hydrogen gas is dissolved. The hydrogen concentration in a hydrogen dissolved liquid is, unrestrictedly, for example, about 1 to about 12 ppm, about 1 to about 10 ppm, and preferably about 1.2 to about 9 ppm, and for example, about 1.6 to about 9 ppm, about 2 to about 10 ppm, about 2 to about 9 ppm, about 3 to about 10 ppm, about 3 to about 9 ppm, about 3 to about 8 ppm, about 4 to about 10 ppm, about 4 to about 9 ppm, about 4 to about 8 ppm, about 5 to about 10 ppm, about 5 to about 9 ppm, about 5 to about 8 ppm, about 6 to about 10 ppm, about 6 to about 9 ppm, about 7 to about 9 ppm, about 8 to about 9 ppm, about 6 to about 8 ppm, and about 6 to about 7 ppm. According to the present invention, when a liquid comprising gaseous hydrogen is administered to a subject, for example, drunk by a subject, hydrogen can be delivered into the body through, for example, gastrointestinal mucosal tissues of the subject.

The kind of liquid is not particularly limited and includes all liquids capable of allowing the gaseous hydrogen to be dissolved therein and applicable to a subject. Examples of the liquid include water, drinks, physiological saline for medical use, injection solutions, intravenous solutions, infusion solutions (may contain therapeutic agents), blood for transfusion, and enteral fluids.

Alternatively, in an embodiment of the present invention, the composition of the present invention in the above liquid forms can also be contained in an infusion drug using the above apparatus. When the composition of the present invention is intravenously dripped into a subject, hydrogen can be delivered to the whole body of the subject through blood stream.

When the composition of the present invention is administered to a subject, for example, by oral administration or parenteral administration, e.g., suction or inhalation, metastasis of a cancer can be significantly suppressed or prevented, or growth of an end stage cancer with metastasis and/or a metastatic cancer(s) therefrom can be significantly suppressed.

As used herein, the term “subject” is vertebrata such as mammals and birds, preferably mammals, and more preferably human. Examples of the mammal include animals including the primates such as human, chimpanzees and monkeys, companion animals such as dogs and cats, and animals for appreciation kept in a zoo. Additionally, examples of the bird include birds for appreciation and companionship.

The term “cancer” used herein may also be called malignant tumor, carcinoma, or malignant neoplasm and is used with an intention to encompass any cancers caused in human. Examples of the cancer include lung cancers, hepatic cancers, pancreatic cancers, stomach cancers, bile duct cancers, colorectal cancers, rectal cancers, bone cancers, prostate cancers, breast cancers, bladder cancers, urothelial cancers, esophageal cancers, brain tumors, ovarian cancers, cervical cancers, lymphomas, tongue cancers, head and neck cancers, osteosarcomas, skin cancers, myelomas, endometrial cancers, pharyngeal cancers, maxillary cancers, oral cancers, lip cancers, thyroid cancers, malignant melanomas, soft tissue tumors, angiosarcomas, undifferentiated soft tissue sarcomas, pediatric solid tumors, and leukemias, but not limited to these cancers. A preferable cancer is a solid cancer.

The term “end stage cancer with metastasis” used herein refers to a primary cancer which is in stage 4 and also has the presence of a metastatic cancer(s) metastasized therefrom detected.

The term “metastatic cancer” used herein refers to a primary cancer caused in a certain organ that infiltrates and migrates to other organs through blood, lymph, or ablation of cancer tissues and fixes thereby to form new blood vessels and grow.

The types of cancers in a primary focus which metastasize to organs and constituent organs and are likely to be metastatic cancers are illustrated below.

For example, a prostate cancer is known to be likely metastasize to the bones and marrow lymph glands, and in the case of bone metastasis, the metastasis is mostly to the bones such as the lumbar spine, pelvis, backbone, and ribs. When a cancer metastasized to bones progresses, cancer cells stimulate the nerves in the bones and cause symptoms such as pains and paralysis, and may trigger hypercalcemia by which bone calcium flows out into blood, and further anorexia, nausea, consciousness disturbance, fracture, and the like are caused.

Organs and constituent organs to which a breast cancer is likely to metastasize include the lymph nodes, lungs, bones, brain, and liver.

Organs and constituent organs to which a colorectal cancer is likely to metastasize include the lymph nodes, liver and lungs.

Organs and constituent organs to which a stomach cancer is likely to metastasize include the lymph nodes, peritoneum, and liver.

Organs and constituent organs to which a lung cancer is likely to metastasize include the brain.

Organs and constituent organs to which an esophagus cancer is likely to metastasize include the lymph nodes, liver, lungs, and bones.

Examples of the kind of lung cancer include small cell lung cancers and non-small cell lung cancers. Small cell lung cancer includes small cell cancers caused at close to the lung entrance. Additionally, non-small cell lung cancer includes adenocarcinomas and large cell cancers caused at the deep part of the lung and squamous cell cancers caused at close to the lung entrance.

The term “suppress growth of an end stage cancer with metastasis and/or a metastatic cancer(s) therefrom” used herein refers to the suppression of cancer growth (or facilitation of regression) and the suppression or inhibition of progress of both cancers in a subject having an end stage cancer with metastasis and/or a metastatic cancer(s) therefrom. At this time, further metastasis can be suppressed by the composition of the present invention.

In an embodiment of the present invention, the composition of the present invention, when is a gas, is sent into the lung by suction, inhalation or insertion through the oral cavity and/or nasal cavity of a subject. At this time, a hydrogen gas inhaler capable of producing hydrogen-containing air/oxygen adjusted to the above gaseous hydrogen concentration at site can be used.

According to the present invention, the gaseous hydrogen in the composition of the present invention is the smallest molecule among all molecules and thus conceived to be able to penetrate blood vessel walls and the like, scavenge the reactive oxygen species produced by a cancer, suppress inflammation induced by the reactive oxygen species, and also suppress or prevent metastasis of cancer cells. Due to these actions such as a scavenge of the reactive oxygen species and suppression effect on inflammation provided by the gaseous hydrogen, the composition of the present invention also suppresses growth of cancers such as metastatic cancers, and further the composition of the present invention prevents precancerous conditions before a cancer is caused, hence conceivably effective for cancer prevention.

Metastasis of a cancer encompasses a condition in which cancer cells in a primary focus enter into, for example, blood vessels and lymphatic vessels, migrate in the streams of blood and lymph, and form cancers again at remote locations. Specifically, metastasis includes hematogenous metastasis, lymphogenous metastasis, disseminated metastasis, and infiltration. It is believed that, depending on an organ or a constituent organ at which a cancer is caused, there are organs and constituent organs to which the cancer is likely to metastasize. As described above, it is known that, for example, a lung cancer is likely to metastasize to lymph nodes, bones, and brain, a colorectal cancer is likely to metastasize to the lung and liver, and an esophagus cancer is likely to metastasize to the lung, liver, and lymph nodes.

The composition of the present invention is effective to suppress or prevent all these metastases. Herein, the prevention of metastasis enables the prevention of recurrence of cancers. Further, the suppression of metastasis is preferably not to cause metastasis in a subject, however, metastasis could occur depending on seriousness of a subject (particularly a human patient), conditions (including complications), constitution, and the like.

The composition of the present invention comprises pulmonarily administering a therapeutically effective amount of gaseous hydrogen under the atmospheric pressure environment or under environment exceeding the atmospheric pressure including a gas containing a therapeutically effective amount of gaseous hydrogen. When a pressure at the time of pulmonary administration exceeds the atmospheric pressure, a pressure can be an atmospheric pressure of 7 or less. For example, a pressure most likely to provide, for example, the suppression and prevention effects on cancer metastasis, or the growth suppression effect on an end stage cancer with metastasis and/or a metastatic cancer(s) therefrom, for a subject can be suitably selected from any pressure of an atmospheric pressure of 5 or less, an atmospheric pressure of 3 or less, an atmospheric pressure of 2 or less, an atmospheric pressure of 1.4 or less, and an atmospheric pressure of 1.3 or less.

When a dose of hydrogen molecules needs to be further increased for a subject to take into the body via the pulmonary administration under the atmospheric pressure environment involving no applied pressure, gaseous hydrogen in a higher concentration can be contained in the composition of the present invention. Alternatively, a dose of hydrogen molecules can also be increased for a subject to take into the body by increasing a flow rate of the composition of the present invention to the subject.

In an embodiment of the present invention, the number of doses of the composition of the present invention is any number of 1 to 3 times a day, and preferably 3 times or more a day. Administration time per dose is any time of 10 minutes to 60 minutes, 60 minutes to 90 minutes, 1 hour to 3 hours, 3 hours to 6 hours, 6 hours to 8 hours, or a maximum of up to 24 hours. The composition of the present invention can be pulmonarily administered to a subject by suction or inhalation. Depending on the subject's symptoms of a cancer such as a lung cancer, pulmonary administration can be carried out for any duration over a period of 1 week to 1 month, 1 month to 6 months, 6 months to 1 year, or 1 year or more.

The composition of the present invention can be pulmonarily administered to a subject using, for example, a tubular cannula or a mask connected to a tube. Additionally, when the composition of the present invention is pulmonarily administered to a subject under the atmospheric pressure environment or under environment exceeding the atmospheric pressure, the composition of the present invention can be pulmonarily administered to a subject by enclosing the subject in a hermetically sealed housing such as a chamber or a capsule loaded with the composition of the present invention or the air.

When a housing as described above is used, the housing is designed in such a way as to have, in addition to an enough size for enclosing a subject, pressure resistance withstandable against an atmospheric pressure of 7 or less. The size of a housing may be a size for enclosing 1 subject, or may be a size for enclosing 2 or more subjects. A housing may also accommodate a bed on which a subject can lie down. A housing can be equipped with an apparatus to measure and display concentrations of gaseous hydrogen, oxygen, and carbon dioxide.

The composition of the present invention in the form of liquid, when administered into the body, has different hydrogen distributions depending on the dosage form. It is conceived that when a liquid comprising gaseous hydrogen is drunk, a distribution to organs including mainly internal organs increases, whereas when gaseous hydrogen is sucked or inhaled or a liquid comprising gaseous hydrogen is intravenously dripped, the hydrogen is distributed by being delivered to the whole body including the brain through blood stream.

The composition of the present invention in the gas form, the composition in the liquid form, and the composition contained in an infusion drug as described above can all be used singly or in combinations of two or more.

The composition of the present invention has the effect of suppressing or preventing metastasis of a cancer and the effect of suppressing growth of an end stage cancer with metastasis and/or a metastatic cancer(s) therefrom, thereby to regress cancers when administered (oral administration or parenteral administration (for example, pulmonary administration, intravenous administration, intraarterial administration, and intraperitoneal administration)) to a subject having a cancer or who is suspected to have a cancer. For this reason, the composition can be used in combination with various cancer therapies (e.g., surgical therapy, chemotherapy, radiotherapy, immunotherapy, or molecularly targeted drug therapy). The method for combination use is not particularly limited and, for example, each of the therapies can be carried out simultaneously or one after the other. However, the composition of the present invention is preferably administered every day.

In an embodiment, a pharmaceutical product for treating a cancer may be added to the hydrogen dissolved liquid. Alternatively, such a pharmaceutical product may be administered separately from the administration of the hydrogen dissolved liquid or a hydrogen gas-containing gas. Example of such a pharmaceutical product include, without limitation, chemotherapeutic agents, molecularly targeted drugs (e.g., antibody preparations), and immunotherapeutic agents (for example, immune checkpoint inhibitors, immunostimulators, and immune cells).

Examples of the chemotherapeutic agent include carboplatin, cyclophosphamide, cisplatin, docetaxel, nedaplatin, paclitaxel, pirarubicin, fluorouracil, bleomycin, mitomycin C, aclarubicin, ifosfamide, irinotecan, etoposide, erlotinib, gemcitabine, epirubicin, eribulin, goserelin, cytarabine, dexamethasone, doxorubicin, mitoxantrone, methotrexate, leuprorelin, vindesine, aclarubicin, oxaliplatin, nimustine, Interferon α, teceleukin, temsirolimus, busulfan, and melphalan.

Examples of the molecularly targeted drug include antibody drugs such as EGFR inhibitors (e.g., cetuximab, afatinib, erlotinib, gefitinib, and panitumumab), ALK inhibitors (e.g., crizotinib), HER2 inhibitors (e.g., trastuzumab and pertuzumab), angiogenesis inhibitors (e.g., bevacizumab, axitinib, sunitinib, sorafenib, pazopanib and regorafenib), EGFR/HER2 inhibitors (e.g., lapatinib), mTOR inhibitors (e.g., everolimus and temsirolimus), BCR-ABL inhibitors (e.g., imatinib, dasatinib, and nilotinib), and membrane differentiation antigen-targeted drug (e.g., rituximab and ofatumumab).

Examples of the immune checkpoint inhibitor include anti-PD-1 antibodies and anti-PD-L1 antibodies. For counteracting against attacks by T cells expressing PD-1 and CTLA4 on the cell surface, cancer cells produce PD-L1 and B7 to escape from the attacks of cytotoxic T cells. The above antibodies bind to PD-1 and PD-L1, or CTLA4 thereby making it easier for T cells to attack cancer cells.

### EXAMPLES

The present invention is further specifically described in reference to the following examples but the technical scopes of the present invention are not limited to these examples.

### [Example 1] Suppression of Cancer Cell Engraftment and Suppression of Metastasis by Hydrogen Gas Inhalation in Cancer Metastasis Model Animals

### 1. Experiment

### Preparation of a Hydrogen-Containing Gas for Inhalation

A hydrogen-containing gas inhaled by mice was prepared by mixing a hydrogen gas and the air. The hydrogen gas was generated by electrolysis of water using hydrogen generator model MHG-2000 (manufactured by MiZ Company Limited, Kanagawa, Japan).

### Measurement of Hydrogen Concentration

A concentration of the hydrogen gas generated by the electrolysis was measured using a hydrogen gas sensor (Nissha FIS, Inc., Hyogo, Japan).

### Mouse Lung Cancer Cell Transplantation Model

Mice were used as the lung cancer cell transplantation models. As cancer cells transplanted to the mice, Lewis Lung Carcinoma cells (LLC), which are a mouse lung cancer cell line commonly used as a lung cancer lung metastasis model in vivo, were used. LLC cells (1.0×10⁴ cells/10 μL) to be transplanted to the mice were prepared by mixing in advance with 10 μL of Matrigel® (CORNING Incorporated) using a pipette.

General anesthesia was induced to 7-week old mice (C57BL/6) by isoflurane to cause complete loss of consciousness and the reaction to pain stimulation. Subsequently, the left chest was incised to inject the total amount (20 μL) of the LLC cells prepared by mixing with Matrigel® to the lung parenchyma from outside the chest cavity to the left lung thereby to carry out the transplantation.

After closing the chest, the anesthesia was stopped to recover the mice. Post-surgery survival and the engraftment of the lung cancer cells were confirmed and then the mice were divided into a hydrogen-inhaled group (5 mice) and a hydrogen-inhalation free control group (4 mice). Hydrogen having a concentration of 3.5% was generated using a hydrogen generator (model MHG-2000) manufactured by MiZ Company Limited to allow the mice to inhale in a chamber in which a pressure was applied to an atmospheric pressure of 1.35.

The hydrogen-inhaled group was allowed to suck hydrogen free air for 7 days since the transplantation of the lung cancer cells and, thereafter from day 8 for over a period of 7 days, allowed to inhale a hydrogen gas for 1 hour a day in a chamber containing the hydrogen gas, and then sacrificed.

On the other hand, the hydrogen gas inhalation free control group was kept in the air for a period of 14 days at the atmospheric pressure without using a chamber, and then sacrificed.

Lung tissues and other tumor sites of the mice from the hydrogen-inhaled group and the control group were harvested to make histological evaluations.

### 2. Results

### Engraftment of Lung Cancer Cells

The engraftment of the lung cancer cells (that is, fixation to lung tissues) was observed using an optical microscope with ×40 magnification and ×200 magnification by producing a paraffin-embedded specimen using the harvested tissues in accordance with a routine method, and then thinly slicing the specimen to prepare a slide specimen, followed by staining with Hematoxylin-Eosin (HE).

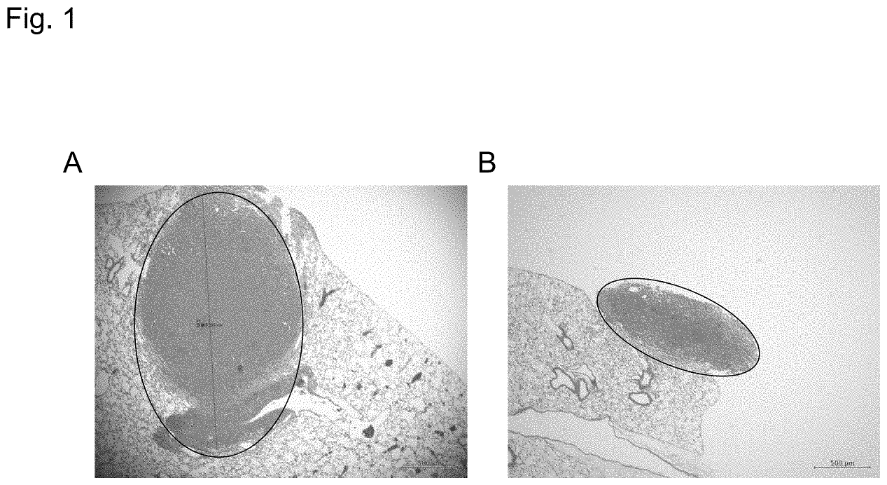

In the control group, all 4 mice to which the lung cancer cells were transplanted had strong engraftment of the lung cancer cells (FIG. 1; the parts enclosed with an oval (parts in an intense dark color)). Three among 4 mice had pathological changes of the cancer in the lung but the remaining 1 mouse had no pathological change in the lung. It is however conceivable that the mouse with which no pathological change was detected possibly had the lung cancer cells transplanted in the chest wall and thus pathological change was not observed in the lung.

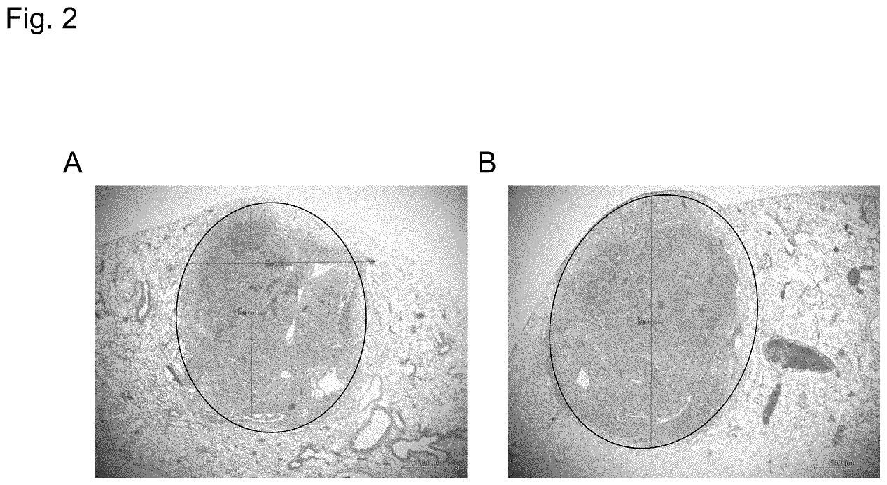

On the other hand, in the hydrogen gas-inhaled group, 3 among 5 mice had weak engraftment of the lung cancer cells (FIG. 2; the parts enclosed with an oval ((note) the color is lighter than the dark parts of FIG. 1.)), and 2 mice had no engraftment of the lung cancer cells detected. This finding, when compared with the results of the control group, indicates that the fixation of cancer cells to the organ is significantly suppressed by the hydrogen gas inhalation.

### Metastasis of Lung Cancer Cells

Metastasis of the lung cancer cells was confirmed by visual observation of the dissected mice.

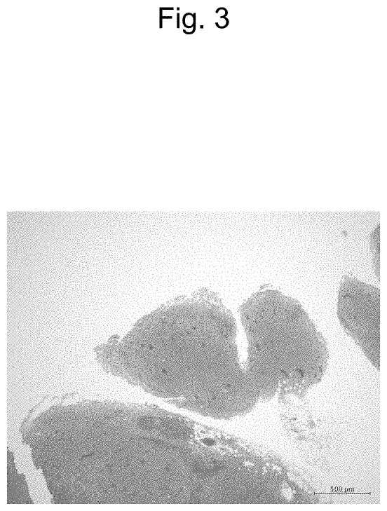

In the control group, 2 among 4 mice had metastasis of the cancer cells. The locations to which the lung cancer cells metastasized include, with overlapping cases, 2 pleural dissemination cases (data now shown) and 1 lymph node metastasis case (FIG. 3; the part in a dark color).

On the other hand, in the hydrogen gas-inhaled group, there was no mouse among 5 mice which had metastasis of the lung cancer cells to other organs. This finding, when compared with the results of the control group, indicates that the metastasis of lung cancer cells is completely or significantly suppressed by the hydrogen gas inhalation.

### Histopathological Observation

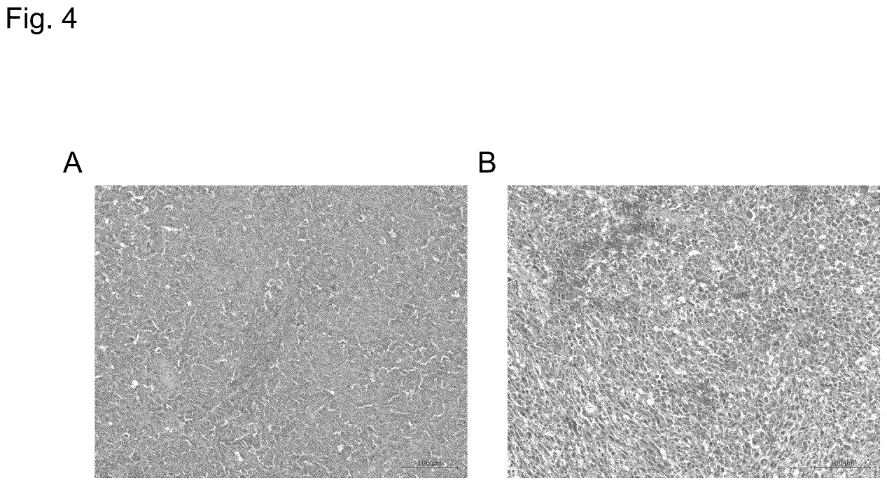

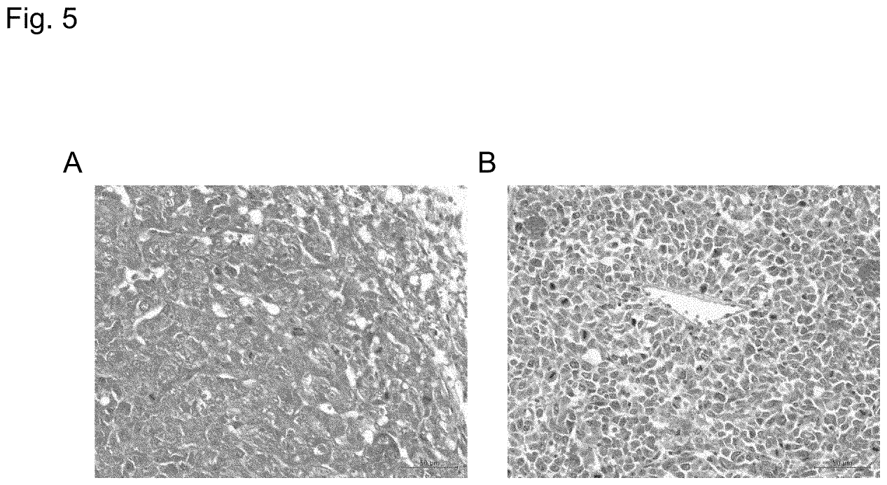

Histopathological specimens of the HE-stained lung tissues were observed using an optical microscope with ×200 magnification (FIG. 4) and ×400 magnification (FIG. 5).

In the control group, tissue images having high cell densities were identified (FIG. 4A and FIG. 5A), whereas in the hydrogen gas-inhaled group, cell densities were comparatively low (FIG. 4B and FIG. 5B). These results, when compared with the results of the control group, indicate that the growth of the lung cancer cells is suppressed by the hydrogen gas inhalation.

In summary, the above experiment results revealed that the hydrogen gas-inhaled group, when compared with the control group, indicated the effect of suppressing the engraftment of the lung cancer cells transplanted to the lung and the evident effect of suppressing the metastasis. Additionally, histopathological examination of the lung parenchyma tissues also suggested the effect of the hydrogen gas inhalation to suppress growth of the lung cancer cells.

### [Example 2] Case Showing Improvement Against Lung Adenocarcinoma by Molecular Hydrogen

(1) History before the hydrogen therapy started

Patient: Japanese female, 71 years old

November 2017: lung cancer was found at medical examination.

Jan. 4, 2018: a 37 mm tumor was confirmed in the lung by simple CT (Compound Tomography) examination. Blood test showed a carcinoembryonic antigen (CEA) value, which is a non-specific adenocarcinoma marker, of 6.2 ng/mL. The reference for normal value of CEA is 5.0 ng/mL.

Feb. 16, 2018: left lower lobectomy was performed. The tumor has grown to 48 mm and surrounding lymph nodes were enlarged, so 5 sites were dissected. Doctors diagnosed that the lung cancer was in stage 3a. CEA was 4.8 ng/mL by the blood test after the patient was discharged.

From Mar. 26, 2018, an anticancer agent therapy by cisplatin and navelbine started. A blood pressure elevated in the first course and the patient's physical conditions declined. A reduction in neutrophil count was detected and the anticancer agent therapy was temporarily suspended.

June 2018: the 4^{th} course of the anticancer agent therapy ended. The hair on the head was lost due to the adverse reaction of the anticancer agents. No vomiting was experienced but the sense of fatigue was intense.

July 2018: thoracic spine defect was confirmed by CT examination. As a CEA value increased to 34.5 nm/mL, a possibility of metastasis was studied.

First half of September 2018: the cancer metastasized to the spine aggravating back pains. Coughing and deep breathing caused pains. Metastases were detected at 3 locations (thoracic spine, lumbar spine, coccyx) on the spine by PET examination.

(2) Continuous hydrogen gas inhalation started

Using hydrogen gas inhaler MHG-2000α manufactured by MiZ Company Limited (hydrogen gas concentration of 6 to 7% by volume), a hydrogen gas was continuously inhaled every day from Oct. 17, 2018 for 2 hours to 3 hours/day.

Nov. 26, 2018: a CEA value decreased to 10 ng/mL.

Dec. 22, 2018: regression of the tumors metastasized to the spine was confirmed by CT examination.

Dec. 27, 2018: a CEA value decreased to 2.6 ng/mL.

Jan. 28, 2019: a CEA value decreased to 1.5 ng/mL.

### [Example 3] Case Showing Growth Suppression of an End Stage Cancer With Metastasis and a Metastatic Cancer Therefrom in an End Stage Cancer Patient by Hydrogen Gas Suction

A patient (Japanese male, in the 70s) diagnosed as having a progressive prostatic cancer in stage 4 with confirmed bone metastasis received, as described below, a therapy by internal medicines (bicalutamide, Flivas OD) for a period of about 2 months since the initial visit. Subsequently, the patient received an anticancer agent therapy (docetaxel IV infusion) and a hormone therapy (LEUPLIN PRO) in combination with administration of the internal medicines.

At the initial diagnosis, a PSA value of 92.5 ng/mL (reference value (normal range) of PSA was 0 to 4 ng/mL) and a Gleason score (malignancy of prostate cancer) of 9 according to the patient's blood test were seriously bad (indicating a metastasized progressive malignant prostate cancer) numerical values. One month after the initial visit, diagnostic results of CT (computed tomography apparatus) and whole body scintigraphy examination confirmed metastases to the pelvis, ribs, and scapulae.

It was 3 days after the initial visit when the patient started the hydrogen gas suction using hydrogen gas generator MHG2000α (manufactured by MiZ Company Limited, hydrogen gas concentration of 6.6% by volume, about 2 L/minute) which produces a mixed gas of hydrogen and the air, and thereafter the patient continued the suction for 60 minutes to 90 minutes substantially every day.

A PSA value at the time of about 1 month after the hydrogen gas suction started decreased to 12.74 ng/mL, a PSA value further about 1 month later was 7.53 ng/mL. At this point (about 2 months after the hydrogen gas suction started), the patient received an anticancer agent infusion (the first time) and further received a hormone injection about 3 months after the hydrogen gas suction started. Subsequently, the patient received the anticancer agent infusion (the second time) about 4 months after the hydrogen gas suction started, further received hormone injections about 6 months and about 12.5 months after the hydrogen gas suction started. At this point, a PSA value was 1.69 ng/mL which decreased to within the reference value. The hydrogen gas inhalation and internal medicine administration were further continued, whereby a PSA value was 1.61 ng/mL about 18 months after the hydrogen gas suction started.

As a result of the whole body scintigraphy examination about 12 months after the hydrogen gas suction started, shade densities at the parts of bone metastasis were a half or less when visually inspected and the parts in the lighter color were substantially vanishing.

The patient still continued the hydrogen gas suction and improved to the extent of functioning at usual work and living a life.

### [Example 4] Case Showing Growth Suppression of Pancreatic Cancer by Hydrogen Gas Inhalation

A patient (Japanese female, 75 years old) diagnosed as having pancreatic cancer (life expectancy of 2 years) in stage 4 continued an anticancer agent therapy for 1 year but stopped the anticancer agent therapy due to adverse reactions to the anticancer agent. Subsequently, metastasis to the liver was detected. The patient, after stopped the anticancer agent therapy, started the hydrogen gas suction using hydrogen gas generator MHG2000α (manufactured by MiZ Company Limited, hydrogen gas concentration of 6.6% by volume, about 2 L/minute) which produces a mixed gas of hydrogen and the air. The hydrogen gas inhalation continually carried out every day for 90 minutes per inhalation, twice a day. At the medical examinations 2 months and 6 months after the inhalation started, there was no change detected in the size of the pancreatic cancer, i.e., the lesion, and the cancers metastasized to the liver. The growth of cancer cells of the pancreatic cancer and the cancers metastasized to the liver was suppressed by the inhalation of hydrogen.

### [Example 5] Case Showing Growth Suppression of Sarcoma by Hydrogen Gas Inhalation

A patient (Japanese female, 38 years old) diagnosed as having undifferentiated soft tissue sarcoma in blood vessels of the heart and lungs had sarcomas of the heart surgically incised, but sarcomas metastasized from the heart to the lungs were located at which blood vessels gathered and hence could not be incised. The patient continued an anticancer agent therapy but stopped the anticancer agent therapy due to adverse reactions. The patient, after stopped the anticancer agent therapy, started the hydrogen gas suction using hydrogen gas generator MHG2000α (manufactured by MiZ Company Limited, hydrogen gas concentration of 6.6% by volume, about 2 L/minute) which produces a mixed gas of hydrogen and the air. The hydrogen gas inhalation was continued every day for 60 minutes to 90 minutes per inhalation, once or twice a day. At the medical examination 3 months after the inhalation started, there was no change detected in the size of lung sarcomas, i.e., the lesion. The growth of sarcomas was suppressed by the inhalation of hydrogen.

### INDUSTRIAL APPLICABILITY

The composition comprising gaseous hydrogen of the present invention is effective to suppress or prevent metastasis of cancers such as a lung cancer, hence medical-industrially useful. Further, the composition of the present invention exhibits not only the effect of suppressing the engraftment of transplanted cancer cells and the evident effect of suppressing metastasis but also the effects of preventing cancer occurrence and suppressing the growth and metastasis of cancers which occurred. Additionally, the composition is also expected to exhibit the effects of preventing the occurrence as well as suppressing growth and metastasis of not only highly metastatic cancer cells and tumors but also low metastatic cancer cells and tumors. Furthermore, the composition is also expected to exhibit the effects of preventing the occurrence and suppressing growth of non-metastatic cancer cells and tumors.

1. A composition for suppressing or preventing metastasis of a cancer, or for suppressing growth of an end stage cancer with metastasis and/or a metastatic cancer(s) therefrom, in a subject having a cancer, wherein the composition comprises gaseous hydrogen as an active ingredient.

2. The composition according to claim 1, wherein the gaseous hydrogen is diluted with air and/or oxygen.

3. The composition according to claim 1, wherein the composition is to be sucked or inhaled by a human or an animal.

4. The composition according to claim 1, wherein the cancer is selected from the group consisting of lung cancers, hepatic cancers, pancreatic cancers, stomach cancers, bile duct cancers, colorectal cancers, rectal cancers, bone cancers, prostate cancers, breast cancers, bladder cancers, urothelial cancers, esophageal cancers, brain tumors, ovarian cancers, cervical cancers, lymphomas, tongue cancers, head and neck cancers, osteosarcomas, skin cancers, myelomas, endometrial cancers, pharyngeal cancers, maxillary cancers, oral cancers, lip cancers, thyroid cancers, malignant melanomas, soft tissue tumors, angiosarcomas, undifferentiated soft tissue sarcomas, pediatric solid tumors, and leukemias.

5. The composition according to claim 1, wherein the subject is a human.

6. The composition according to claim 1, which is used in combination with any of other cancer therapies.

7. A method for suppressing or preventing metastasis of a cancer in a subject, comprising administering the composition according to any one of claims 1 to 6 to the subject having a cancer.

8. A method for suppressing growth of an end stage cancer with metastasis and/or a metastatic cancer(s) therefrom in a subject, comprising administering the composition according to any one of claims 1 to 6 to the subject having an end stage cancer with metastasis and/or a metastatic cancer(s) therefrom.
